(19) United States
(12) Patent Application Publication
(10) Pub. No.: US 2005/0042279 A1
(43) Pub. Date: Feb. 24, 2005

(54) Pharmaceutical formulation comprising more than 15% tamoxifen

(76) Inventor: Marcel De Matas, Macclesfield (GB)

Correspondence Address: ROPES & GRAY LLP, ONE INTERNATIONAL PLACE, BOSTON, MA, 02110-2624, US

(21) Appl. No.: 10/493,166

(22) PCT Filed: Oct. 23, 2002
(86) PCT No.: PCT/GB02/04835

(30) Foreign Application Priority Data
Oct. 24, 2001 (GB) 0125492.9

(51) Int. Cl. A61K 9/20; A61K 31/205

(52) U.S. Cl. 424/464; 514/554

(57) ABSTRACT

The invention provides a pharmaceutical formulation comprising >15% tamoxifen (w/w). In a further embodiments of the invention the invention provides said formulation further comprising from about 0.45% to 1% (w/w) anastrozole. The invention also provides said formulation in the form of a tablet and processes for the preparation of said formulation.

This application is a national stage filing under 35 U.S.C. 371 of International Application No. PCT/GB02/04835, filed Oct. 23, 2002, which claims priority from United Kingdom Patent Application No. 0125492.9, filed Oct. 24, 2001, the specification of which is incorporated by reference herein. International Application No. PCT/GB02/04835 was published under PCT Article 21(2) in English

Tamoxifen is an anti-estrogen used in the treatment and prevention of breast cancer. A challenge facing workers for making a solid dose formulation of tamoxifen is in providing a formulation having a high percentage of tamoxifen citrate (e.g., >15% w/w). Further challenges are in providing a low dose of an aromatase inhibitor such as anastrozole in combination with a high percentage of tamoxifen. Provided herein is a pharmaceutical formulation comprising >15% tamoxifen by weight (w/w), preferably up to 23%. Thus, according to the invention there is provided a pharmaceutical formulation comprising >15% of tamoxifen further comprising:
a) an effective amount of a diluent;
b) an effective amount of a disintegrant;
c) an effective amount of a binder; and
d) an effective amount of a lubricant;
   wherein said formulation comprises granules having a moisture content of less then or equal to 2% w/w and having a granule surface area of from about 30,000 to about 55,000 cm²/100 g. In reference the percentages “(w/w)” refers to the mass of a component in ratio to the mass of the total amount of the formulation unless otherwise noted. Tamoxifen citrate is preferred but the formulation described herein also applies to other salts of tamoxifen and to the equivalent amount of free base. tamoxifen may be provided up to about, by weight, 23% (w/w). Preferably, Tamoxifen may be provided in the range 16% to 20% (w/w), most preferably between 16.5% and 17.5% (w/w). Tamoxifen may be provided in the formulation in combination with an aromatase inhibitor. Aromatase inhibitors that may be used in the current invention are, for example, anastrozole, formestane, atamestane, letrozole, pentrozole, dadrozloze, or vorozole. In a surprising aspect of the invention, a formulation provides, at a low concentration of the aromatase inhibitor, good content uniformity for the aromatase inhibitor. Anastrozole may be provided herein in a low concentration, for example, in an amount from about 0.45% to about 1% (w/w), preferably 0.45% up to about 0.80% (w/w), yet more preferably from about 0.5% up to about 0.67% (w/w), more preferably from 0.50% up to about 0.70% (w/w) and most preferably at 0.56% (w/w). Anastrozole is a potent and selective non-steroidal aromatase inhibitor. It is used for example in the treatment of advanced breast cancer in postmenopausal women with disease progression following tamoxifen therapy.

The pharmaceutical formulation may be provided with the active ingredients in combination with other active agents. Other ingredients may also be provided in the pharmaceutical formulation. These ingredients may be provided having specifications set out for example in the 2001 European Pharmacopeia (“PhEur”). The formulation may comprise an effective amount of a binder, for example in an amount, by weight, from 1 to 4% (w/w). Suitable binders for use in the present invention include polymeric binders such as povidone and cellulose binders such as hydroxypropyl methylcellulose. A preferred binder is povidone. The formulation may further comprise an effective amount of a disintegrant, for example in the amount, by weight, of about 2% to about 5% (w/w). Disintegrants suitable for use herein include, for example, sodium starch glycollate and crospovidone. A preferred disintegrant is croscarmellose sodium. The formulation may further comprise an effective amount of at least one diluent, for example in the amount, by weight, of about 60% to about 80% (w/w). If two diluents are provided the first diluent may be provided in the amount, by weight, of from about 50% (w/w). Suitable diluents that may be used include lactose monohydrate and microcrystalline cellulose. A preferred first diluent is lactose monohydrate. Lactose monohydrate is preferably provided in combination with microcrystalline cellulose in an amount, by weight, of about 55% to about 75%, preferably about 65% and about 5% to about 20%, preferably 10% (w/w) respectively. Combinations of dicalcium phosphate dihydrate and microcrystalline cellulose or mannitol with microcrystalline cellulose also may be used. The formulation may also comprise an effective amount of a lubricant, for example in the amount, by weight, from about 0.5 to 2% (w/w). Lubricants that may be used herein include fatty acids and their salts. Suitable lubricants that may be used include magnesium stearate and stearic acid, preferably magnesium stearate.

Further examples of tablet excipients are given in the Handbook of Pharmaceutical Excipients (3^{rd} Edition, 2000), Editor: Kibbe, Publisher: American Pharmaceutical Association, which is incorporated herein by reference.

These ingredients are used to make granules which may be compressed to form tablet cores or used in other formulations such as in a capsule. The granules are preferably compressed to form a tablet core. The core may be coated for example by typical coating processes. The coating may comprise an effective amount of a film former, for example hydroxypropylmethylcellulose; an effective amount of a plasticiser, for example a low molecular polyethylene glycol such as polyethylene glycol 300; and an effective amount of an opacifier, for example titanium dioxide.

The ingredient may be provided in an amount effective to provide a content uniformity of the active agents of 6% RSD (relative standard deviation). For example, anastrozole in the amount of 0.90 mg to 1.1 mg and tamoxifen free base equivalent in the amount of 18 mg to 22 mg both had measured RSD of ≦6% or better in tablets made according to the formulations and methods described herein. The ideal content uniformity for each drug substance is preferably <2% RSD and can be achieved using the formulation and methods described herein.

Further provided herein is a method of making a pharmaceutical formulation of tamoxifen comprising: 1) mixing tamoxifen, anastrozole, lactose monohydrate, microcrystalline cellulose, croscarmellose sodium and povidone, to form a dry mixture; 2) mixing the dry mixture with water to form a wet mass wherein the water may be added to the dry mixture for instance by spraying the water onto the dry mix; 3) breaking up large aggregates, for example by passing through a screen; 4) drying the granules to a moisture content of less than or equal to 2% (w/w) to form dry granules; 5) milling the dry granules to form milled dry granules; 6) adding a lubricant to the milled dry granules; 7) blending the milled dry granules containing the lubricant; and 8) compressing the blend into tablets.

Also provided herein is a method for making a tamoxifen formulation comprising: 1) charging ingredients, comprising tamoxifen, anastrozole, at least one diluent, a disintegrant, and a binder to a mixer; 2) mixing the ingredients to form a mixture of ingredients having uniform distribution; 3) adding water to the mixture of ingredients to form a wet mass; 4) passing the wet mass through a screen; 5) drying the granules to form dried granules having a moisture content of less than or equal to 2% (w/w); 6) adding a lubricant to the dried granules to form a lubricant:dried granule mixture; 7) milling the lubricant:dried granule mixture; and 8) blending the milled mixture to form blended granules. In an alternative embodiment of this process, the lubricant can be added after milling such that the granules and lubricant are milled separately as and then the lubricant added to the granules in replacement of steps 6) and 7) above.

The granules may be used for example in capsules and tablets. For tablets the method comprises further compressing the blended granules to form tablet cores. The methods described above may further comprise applying a film coat wherein said film coat is made by mixing a film former, a plasticiser, an opacifier, and water.

The formulations and methods provided herein are useful for treating patients with breast cancer, preferably early stage breast cancer, by administering a formulation described herein to a patient.

A dry mix may be prepared by adding some of the ingredients described above, i.e tamoxifen, anastrozole, lactose monohydrate, microcrystalline cellulose, croscarmellose sodium and providone. Preferably, the ingredients are provided in layers such as by symmetrical addition, The ingredients are preferably added in a specific order for example, a dry mix may be charged to a vessel as follows: 3/10 first diluent, all of the binder and the second diluent, 2/10 of the first diluent, ½ of the tamoxifen citrate, all of the anastrozole, ½ of the tamoxifen, 2/10 of the first diluent, all of the croscarmellose sodium, and the remainder of the first diluent.

Formulations containing 4% w/w croscarmellose sodium were made by adding the disintegrant intra-granularly and extra-granularly. Formulations were made containing disintegrant which was added intragranularly and formulations were made containing half of the disintegrant intragranularly and half extragranularly. The extragranular material was added during the lubricant blending stage. Crushing strength and disintegration time was measured for a composite sample of tablets from each batch which were taken during compression. Negligible differences in tablet crushing strength, disintegration time and granule flow was observed for formulations made using intra and extragranular disintegrant. Hence, it was shown that the addition of a disintegrant in a single step can be used without any detrimental effects to the tablet and granules physical properties.

The dry mix described above should be mixed for a time sufficient to achieve a content uniformity of anastrozole having a measured relative standard deviation (RSD)≦6% or better. The equipment that may be used for the dry mixing step is preferably a high shear mixer granulator. High impeller speeds may improve the content uniformity and homogeneity for both ingredients.

For example, good content uniformity (RSD<2%) is found over a range of batch sizes (e.g., at the 5.4 kg and 18 kg mix size) with a mixing time of 8 and 10 minutes respectively and where ingredients were charged to the mixer in layers. Charging the ingredients to the mixer as described above results in a material that has good content uniformity for both tamoxifen and anastrozole.

In carrying out the method of the invention it is preferable to add the binder (e.g., povidone) during the dry mix stage.

In carrying out the wet granulation step of the methods described herein, water is typically added to the dry mix in an amount between 53 mg/tablet (299 g/kg of dry mix constituents) to about 80 mg/tablet (449 g/ kg of dry mix constituents) preferably at about 67 mg/tablet. At 53 mg/tablet a finer material is obtained (54% to 71% of the granules <125 μm) while still having good flow characteristics with some filming of the tablet punches. Water provided at levels of 80 mg/tablet produced coarse granules having excellent flow but the tablets formed from these granules had reduced hardness and prolonged disintegration times.

Extending the wet mixing time by greater than 50% for a batch manufactured using the optimal level of water, reduces the compressibility of granules. A total wet mixing time of 7.5 minutes is preferably used for 18 kg batches. Wet mixing is preferably done at slow impeller speeds.

Wet granules may be passed though a screen containing square apertures, preferably about 0.375 inches or greater, preferably up to about 0.5 inches. In selecting an appropriate aperture size the goal is to produce material which can be easily fluidized in the fluid bed drier (as assessed visually) to avoid wet mass accumulation and inefficient drying. The wet granules are dried, preferably after breaking up large aggregates, to a moisture content of ≦2% w/w. Granules dried to the levels described above give a preferred compressibility, e.g., a crushing strength of about 6-12, preferably 7-11 Kp (kiliponds), most at about 9 Kp.

The dried granules may be milled, preferably using a screen having an aperture 0.062 inches or larger.

A lubricant may be added to the milled dry granules. The lubricant, such as magnesium stearate, may be milled by passing it through a screen. The aperture of the screen is preferably about 0.041 inches. The general purpose of the milling is to de-lump the magnesium stearate. The milled dry granules may be mixed.

The granules, preferably containing the lubricant, are blended and the granules may be compressed into tablets preferably with a hardness described above (6 kp or 12 kp preferably with a disintegration time in water of ≦10 minutes (specific protocols can be found in the US Pharmacopoeia and the European Pharmacopoeia). Granules having a surface area within the range of about 35,000 to about 50,000 cm²/100 g and exhibit good compressibility. In carrying out certain methods of the invention it is preferable to apply pre-compression which increases the crushing strength of the tablets and helps to prevent capping.

The preferred surface area, when used in the formulation, for tamoxifen citrate is greater then 0.95 m²/g and is from 0.5 to 1.5 m²/g for anastrozole.

The tablet described above may be coated. Suitable coatings can be prepared from concentrates, such as Opaspray White M-I-28813 and White Speedpaste 30001 (D.F. Ansteads Ltd.). For example White Speedpaste 30001 comprises 33.3% (w/w) titanium dioxide, 2% (w/w) hydroxypropylmethylcellulose (HPMC 606) and 10% (w/w) industrial methylated spirit (IMS) as a preservative. Additional ingredients added during preparation of coating mixtures from concentrates include: a suitable plastisizer such as polyethylene glycol 300 and a film former, such as hydroxypropylmethylcellulose. This white film coat may be added to the tablets using for example perforated drum coaters.

The film coat may be applied using conventional perforated drum coaters. For example at scales of approximately 10 kg and 50 kg anastrozole/tamoxifen citrate combination tablets have been shown to demonstrate good stability at 6 months at 25° C./60% Relative humidity (RH), 6 months at 40° C./75% RH and 6 months at 50° C. (ambient humidity). For anastrozole, no more than 0.2% degradation was observed and for tamoxifen citrate, no more than 0.7% degradation was observed.

The invention will now be exemplified with reference to the following non-limiting example.

### EXAMPLE 1

Tablets were made comprising a combination of anastrozole and tamoxifen citrate with the ingredients as set out in Table 1 which follows.

TABLE 1
Composition of anastrozole/tamoxifen Citrate white film coated tablets.

| | Compendial | Quantity |
|---|---|---|
| Ingredient (tablet core) | designations | (mg/tablet) |
| Tamoxifen Citrate | PhEur, USP, JP | 30.4 |
| Anastrozole | — | 1.0 |
| Lactose Monohydrate (450 mesh) | PhEur, USNF, JP | 118.0 |
| Microcrystalline Cellulose (Avicel | PhEur, USNF, JP | 18.0 |
| PH101) | | |
| Croscarmellose Sodium | PhEur, USNF, JP | 7.2 |
| Povidone (K29-32) | PhEur, USP, JP | 3.6 |
| Magnesium Stearate | PhEur, USNF, JP | 1.8 |
| Purified Water^{a} | PhEur, USP, JP | 67.0 |
| Nominal tablet core weight | | 180.0 |
| | | Quantity |
| Ingredient (film coat) | | (mg/tablet) |
| Hydroxypropyl Methylcellulose 2910 | PhEur, USP, JP | 2.70 |
| (6 cps) | | |
| Polyethylene Glycol 300 | PhEur, USNF, JP | 0.54 |
| Opaspray White (M-1-28813)^{b} | | 2.70 |
| Purified Water^{a} | PhEur, USP | 30.0 |
| Nominal coated tablet weight | | 184.19 |
| ^{a}Purified water was used as the granulating fluid during the manufacture of the tablet core and is removed during granule drying. Purified water is also used as | | |
| # the solvent/carrier fluid during film-coating and is removed during the coating process. The quantities of purified water may be modified to accommodate processing requirements. | | |

^{b}Opaspray white (M-1-28813) is a proprietary product supplied by Colorcon Ltd., Dartford, Kent, UK which provides titanium dioxide (0.9 mg/tablet) and HPMC (0.05 mg/tablet).

A 54 kilogram batch was prepared by preparing three 18 kg mixes and combining them at the blending stage. The batches were made as follows: A dry mix of the ingredients from Table I, were charged to a bowl of a mixer granulator in the following order: ¼ Pharmatose® lactose monohydrate (DMV International, Veghel, The Netherlands), Plasdone® povidone (International Speciality Products, New Jersey, USA), Avicel® PH-101 microcrystalline cellulose (FMC International, Philadelphia, Pa., USA), ¼ lactose, ½ tamoxifen citrate, anastrozole, ½ tamoxifen citrate, ¼ lactose, Ac-Di.Sol® croscarmellose sodium (FMC International, Philadelphia, Pa., USA) and ¼ lactose. The levels specified were approximate and were adjusted to simplify the weighing and charging procedure. The powdered ingredients were mixed for 10 minutes in a fixed bowl mixer-granulator. A fast impeller speed was used (e.g., 350 rpm) with no chopper. Purifed water was transferred (66.5 mg/tablet, 373 g/kg of dry mix constituents) to the pressure tank. The water was sprayed on to the dry mix over a duration of approximately 5.5 minutes using the impeller at slow speed (e.g., about 200 rpm). After 2 minutes of mixing, the chopper was turned on at slow speed (e.g., 1500 rpm). After all the water was added and the mix inspected, any material adhering to the sides of the bowl or the mixing blades was dislodged. The dry-mix was further mixed to produce a wet mass of a medium consistency, adding water when necessary. The total wet mixing time was about 8 minutes: The dry mix contained drug substance at levels of 16.9% w/w for tamoxifen citrate and 0.56% w/w for anastrozole. Good content uniformity was obtained as determined by HPLC.

The wet granules were passed through a rotary impeller screening mill such as a Comil (Ytron Quadro, Chesham, Bucks, UK) using a 0.375″ square aperture screen. The wet granules were transferred to a fluid bed drier and dried to a moisture content of ≦2% w/w as determined by the loss on drying method.

Magnesium stearate (Mallinckrodt, St Louis, Mo., USA) was added to the dried granules which were passed through a 0.039 inch screen attached to a Comil.

All three 18 kg mixes were combined and blended for 3 minutes in a V-blender. The tablets were compressed using an 8 mm, round, standard concave plain, chromium tipped tooling using a rotary tablet press, such as a Manesty Betapress (Manesty Ltd., Liverpool, UK).

The tablets were coated with a white film coat, prepared using Opaspray White M-I-28813 and the ingredients are set forth in Table I. The above formulation produced good quality tablets with crushing strengths of 7-10 kp and disintegration times of 5-9 minutes. The tablets had a friability of less than 0.2%. The content of anastrozole and tamoxifen was 0.99 mg/tab (1.74% RSD) and 20.61 mg/tab (1.97% RSD) respectively. Dissolution testing showed that at least 85% of each active agent was released within 30 minutes using a USP2 (US Pharmacopoeia) dissolution apparatus using a paddle speed of 75 rpm and in 1000 ml 0.02 molar HCl, (pH 1.8).

1. A pharmaceutical formulation comprising >15% of tamoxifen and
a) an effective amount of a diluent;
b) an effective amount of a disintegrant
c) an effective amount of a binder; and
d) an effective amount of a lubricant;
wherein said formulation comprises granules having a moisture content of less than or equal to 2% (w/w) and having a granule surface area of from about 30,000 to about 55,000 cm ²/100 g tamoxifen.

2. A pharmaceutical formulation as defined in claim 1, further comprising an effective amount of anastrozole.

3. A pharmaceutical formulation as defined in claim 1 or 2 wherein the formulation is a tablet.

4. A pharmaceutical formulation as defined in claim 3, further comprising a coating comprising:
an effective amount of a film former;
an effective amount of a plasticiser; and
an effective amount of an opacifier.

5. A pharmaceutical formulation as defined in claim 3 further comprising a coating comprising:
a) about 2.75 mg hydroxypropylmethylcellulose;
b) about 0.54 mg Polyethylene glycol 300; and
c) about 0.90 mg titanium dioxide.

6. A pharmaceutical tablet comprising:
from about >15% to about 20% (w/w) tamoxifen citrate;
from about 0.5% to about 0.67% (w/w) anastrozole;
from about 1% to about 4% (w/w) of a binder;
about 2 to 5% (w/w) disintegrant; and
about 0.5 to 2% lubricant.

7. The tablet as defined in claim 6, wherein the tablet further comprises a first diluent in an amount from about 60% to about 80% (w/w).

8. The tablet as defined in claim 7, where the tablet further comprises a second diluent in an amount from about 5 to 20% (w/w), wherein the total amount of both diluents comprises about 60 to 80% (w/w) of the total weight of the tablet.

9. A tablet consisting essentially of:
a) about 30.4 mg tamoxifen citrate;
b) about 1.0 mg anastrozole;
c) about 118.0 mg lactose monohydrate;
d) about 18.0 mg microcrystalline cellulose;
e) about 7.2 croscarmellose sodium;
f) about 3.6 mg povidone; and
g) about 1.8 magnesium stearate.

10. A method for making a pharmaceutical formulation of tamoxifen citrate comprising:
a) dry mixing tamoxifen citrate, anastrozole, lactose monohydrate, microcrystalline cellulose, croscarmellose sodium and povidone to form a dry mixture;
b) mixing the dry mixture with water to form a wet granulation;
c) breaking up large aggregates;
d) drying the granules to a moisture content of less than or equal to 2% w/w to form dry granules;
e) milling the dry granules to form milled dry granules;
f) adding a lubricant to the milled dry granules; and
g) blending the dry granules containing the lubricant.

11. A method of making a pharmaceutical formulation comprising:
a) charging ingredients to a mixer wherein said ingredients comprise tamoxifen citrate, anastrozole, at least one diluent, a disintegrant, and a binder to a mixer;
b) mixing the ingredients to form a mixture of ingredients having uniform distribution;
c) adding water to the mixture of ingredients to form a wet mass;
d) passing the wet mass through a screen to form granules;
e) drying the granules to form dried granules having a moisture content of less than or equal to 2% w/w;
f) adding a lubricant to the dried granules to form a lubricant:dried granule mixture;
g) milling the lubricant:dried granule mixture; and
h) blending the milled mixture to form blended granules.

12. The method as defined in claim 10 or 11 further comprising compressing the granules into a tablet.

13. The method as defined in claim 12, further comprising applying a film coat to said tablet.
